(19) United States
(12) Patent Application Publication
(10) Pub. No.: US 2021/0324458 A1
(43) Pub. Date: Oct. 21, 2021

(54) ENHANCED SIGNAL TO NOISE RATIOS FOR PCR TESTING WITHIN A FRET DOPED NANO-STRUCTURED CERAMIC FILM

(71) Applicant: Nanopec, Inc., Tucson, AZ (US)

(72) Inventor: Mario Blanco, Tucson, AZ (US)

(73) Assignee: Nanopec, Inc., Tucson, AZ (US)

(21) Appl. No.: 17/231,904

(22) Filed: Apr. 15, 2021

Related U.S. Application Data
(60) Provisional application No. 63/010,268, filed on Apr. 15, 2020.

(51) Int. Cl. C12Q 1/6848; C12Q 1/686

(52) U.S. Cl. CPC C12Q 1/6848; C12Q 1/686

(57) ABSTRACT

A nano-structured ceramic film engineered for the study of biological samples. The films have a plurality of pores with a narrow and ordered pore-size distribution with pore diameters between 50 nm to 400 nm (±10%). The films are doping with metal compositions that provide fluorescence resonance energy transfer (FRET) capabilities with significant fluorescence signal enhancement and low noise. A hairpin DNA construct with a quencher and fluorophore is covalently linked to the surface (including inside the pores) of the ceramic film and configured to react to any target sequences in solution, resulting in separation of the quencher-fluorophore pair. The chelated metal ion FRET centers doped within the nano-structure ceramic film provider for long lasting fluorescence signals with less noise.

### CROSS-REFERENCE TO RELATED APPLICATION

The present application claims priority to U.S. Provisional App. No. 63/010268, filed on Apr. 15, 2020.

### BACKGROUND OF THE INVENTION

### 1. Field of the Invention

The present invention relates to nanostructured ceramic films and, more specifically, to a FRET doped ceramic film providing enhanced signal to noise ratios for polymerase chain reaction (PCR) testing applications.

### 2. Description of the Related Art

PCR testing is widely used to identify the presence of a pathogen or a gene in a biological sample. Specific DNA sequences are amplified through the polymerase chain reaction (PCR) quantitatively (qPCR) and in real time (RT-PCR). The presence of a sought after sequence, or sequences, is determined through hybridization of the amplified DNA with fluorescence molecules built into cDNA single strands, the probes. The amount of probe fluorescence is proportional to the amount of the DNA sought sequence in the sample. Low levels of fluorescence, below a certain threshold are flagged as negatives. Although proven to be more effective than other testing bio-assays, such as immunofluorescence (IF) which detects proteins such as antibodies or biomarkers, it is not free from failures due to low signal to noise ratios. For example, fluorescence in-situ hybridization (FISH) or its variant utilizing DNA hairpins probes to detect single-point mutations are reported to suffer from low signal output when grafted on glass, limiting their usability. The hair pins contain a fluorescence quencher which inhibits the fluorescence while the probe is not hybridized.

Effective means to overcome low signal to noise ratios (SNR) in PCR testing are needed as a low SNR results in high rates (>5%) of false negatives in disease and genetic testing. Particularly troublesome is the high rates of false negatives (>30%) in cancer and viral (COVID-19) testing well documented in the scientific literature. Often these false negatives may be due to operator error in sample manipulation steps, contaminated or expired reagents, to low instrument sensitivity or low levels of the sought after DNA sequence due to early disease onset. A significant amount of effort has gone into reducing the levels of noise to increase SNR values, however, these efforts have only been partly successful and signal noise continues to be a problem. Accordingly, there is a need in the art for an approach that can provide enhanced signals for PCR testing.

### BRIEF SUMMARY OF THE INVENTION

The present invention comprises nano-structured ceramic films that are engineered to provide a favorable substrate for the study of biological samples by focusing on the enhancement (amplification) of the signal but with only low added noise levels. The ceramic films of the present invention have a narrow and ordered pore-size distribution with pore diameters between 50 nm to 400 nm (±10%), resulting in a high and predictable amount of increased surface area from conventional surfaces (>100 fold) and a hydrophilic surface that is superior to glass. More specifically, the films are used to form a substrate for biological detection by providing a ceramic film having a plurality of pores formed in a surface of the ceramic film. Key to the invention is the use of a fluorescence resonance energy transfer center having a chelated metal ion is embedded inside the ceramic film. A hairpin nucleic acid construct is covalently linked to the surface of the ceramic film. The hairpin nucleic acid construct is a nucleic acid strand coupled to a fluorophore at one end and a quencher at an opposing end. The hairpin nucleic acid construct also comprises a linker coupled to the fluorophore and to the surface of the ceramic film. The hairpin nucleic acid construct corresponds to a target nucleic acid sequence such that the target nucleic acid construct will hybridize with the nucleic acid strand. The plurality of pores are arranged in a honeycomb pattern. Each of the plurality of pores has a diameter between 50 nm and 400 nm within a tolerance of ten percent.

The present invention also comprises a method of detecting the presence of a target nucleic acid strand. The method first comprises the step of providing a ceramic film having a plurality of pores formed in a surface of the ceramic film and a fluorescence resonance energy transfer center having a chelated metal ion embedded in the ceramic film, and a hairpin nucleic acid construct covalently linked to the surface of the ceramic film and comprising a complementary nucleic acid strand coupled to a fluorophore at one end and a quencher at an opposing end. The method also comprises the step of exposing the ceramic film to a solution that may contain the target nucleic acid sequence. The method last comprises the step of measuring a fluorescence signal from the ceramic film to determine whether the solution containing any of the target nucleic acid sequence during DNA amplification steps common to a PCR diagnostic test.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a graph of typical pore size distribution obtained from processing digitally a scanning electron micrograph of a ceramic disc prepared according to the present invention, where the standard deviation of the pore distribution is approximately σ=10 nm;

FIG. 2A is a schematic of a nano-structured ceramic film according to the present invention with doped with FRET centers;

FIG. 2B is a diagram of a FRET center for use in the ceramic film of the present invention that comprises a metal ion chelated by organic molecules, such as three oxalate anions;

FIG. 3 is a schematic showing DNA hairpins as highly specific molecular detection tools for use with the ceramic film of the present invention; and

FIG. 4 is a schematic of a FRET doped ceramic film according to the present invention;

FIG. 5 is a graph of a dye commonly used with PCR (fluorescein) emission for two ceramic examples of a material incorporating a transition metal (Al) and a Lanthanide (Tb) where the dotted line corresponds to a standard glass bottom microplate well for comparison purposes;

FIG. 6 is a graph of another commonly used PCR fluorescent probe dye, rhodamine-6G, emission for two ceramic examples of a material incorporating a transition metal (Al) and a Lanthanide (Eu) where the dotted line corresponds to a standard glass bottom microplate well;

FIG. 7 is a table of a dye commonly used as a standard and for testing purposes in the presence or absence of DNA probes, Alexa Fluor 488, AB150113, goat poly clonal antibody to mouse at an initial concentration of 2 μg/ml has been diluted and the enhancement factors recorded for two examples of MetaFluorex (trademark) using the current invention where the final concentration of Alexa Fluor is 3.9 ng/ml; and

FIG. 8 is a chart of absolute fluorescence counts per second for various concentrations of AlexaFluor 488 fluorescent dye using the present invention (MetaFluorex) and a standard glass bottom well.

### DETAILED DESCRIPTION OF THE INVENTION

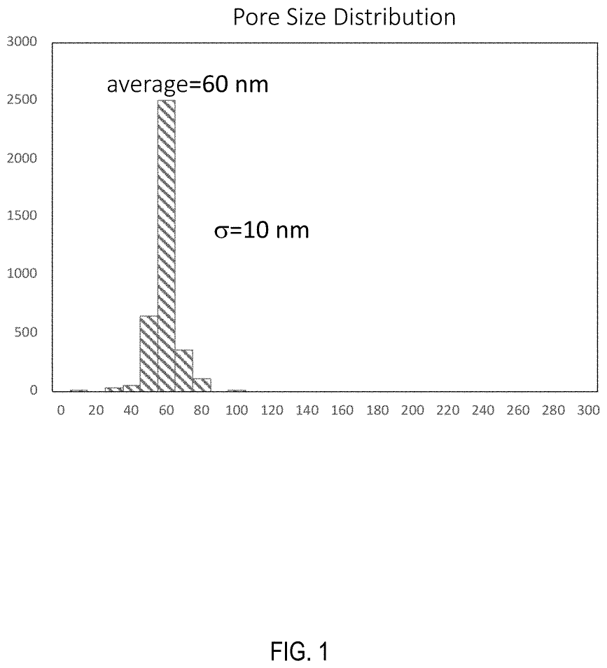

Referring to the figures, wherein like numeral refer to like parts throughout, the present invention comprises nano-structured ceramic films engineered to provide a favorable substrate for the study of biological samples. As seen in FIG. 1, films according to the present invention have a narrow and ordered pore-size distribution. The nano-structured ceramic films have pore diameters between 50 nm to 400 nm (±10%), which provides high, predictable, amounts of increased surface area (>100 fold over conventional surfaces) and a hydrophilic surface that is superior to glass, thereby offering an ideal environment to study living biological samples.

The present invention further comprises metal doping compositions that provide fluorescence resonance energy transfer (FRET) capabilities with significant fluorescence signal enhancement and low noise. Enhancement factors greater than 20× are possible (2000% increase in fluorescence over glass substrates) with only minor levels of added noise due to the presence of the FRET centers (<5%) as excitation and detection wavelengths are purposefully set apart from one another.

The present invention also comprises a hairpin DNA construct covalently linked to the surface (including inside the pores) of a FRET modified ceramic film which serves as the fluorescent probe to detect the presence of the amplified DNA sequence of interest. The probe grafting process is performed through available ceramic surface modification chemistry, forming an extremely durable, thermally stable bond between the ceramic surface and the DNA constructs. Hairpin structures acceptable for use with the present invention are often referred to as molecular probes and are commonly found in solution or linked to polymeric beads, glass, silicon and other solid substrates. However, as these conventional substrates are photo-inactive, none of these substrates participate in the fluorescent process and usually only serve as a mechanical means to support the fluorescent probes. The hairpin DNA constructs of the present invention comprise hairpin DNA fluorescent probes that that are covalently linked to a photo-active substrate, i.e., a chelated metal ion doped nano-structured ceramic film according to the present invention, to enhance fluorescent signals in a significant way.

Typical fluorescence lifetimes in PCR detection are measured in the range of 10 to 100 nanoseconds. Enabled by chelated metal ion FRET centers doped within the nano-structure ceramic film, the present invention provides long lasting, several micro-seconds, fluorescence signals. The chelated metal ion centers include transition metals and lanthanides that are embedded in the ceramic film during fabrication at low concentration (doping). Due to the absence of quenching solvent water molecules, the FRET centers can remain in a long-lived triplet state (on the order of tens of microseconds) when excited by an external source. This in turn makes them a convenient energy reservoir—the FRET centers act as non-radiative (electronic) donors, amplifying the fluorescence signal strength by recharging the fluorescent probes over a period of time comparable to one thousand times their normal fluorescent lifetimes (tens of nanoseconds). This amplification effect is only effective on a nanometric-scale, which has been established to be around 10 nm. The maximum amount of doping is critical to avoid self-quenching. The preliminary results collected using RT-PCR DNA amplification augmented by electro-optical means have shown fluorescence amplification, compared to standard (glass, plastic) substrates, values >1000%, with only a minor (<5%) increase in background noise. Such fluorescence enhancement corresponds to improvements of signal-to-noise ratio to ten or more (SNR >10).

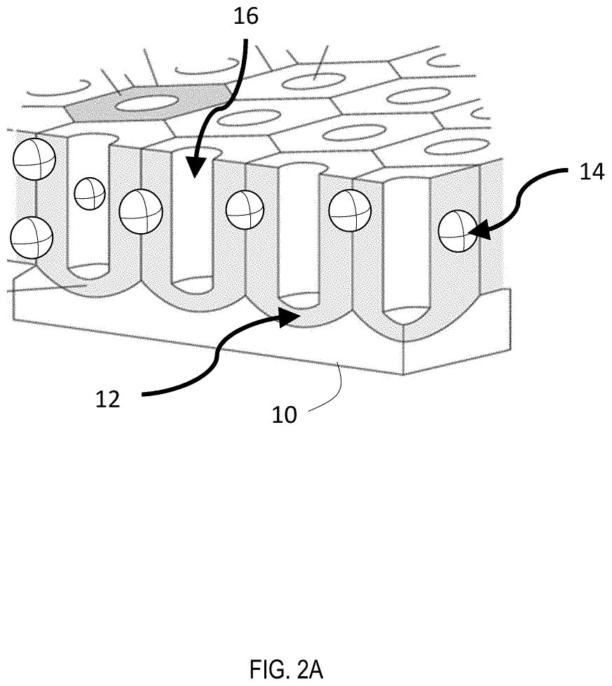

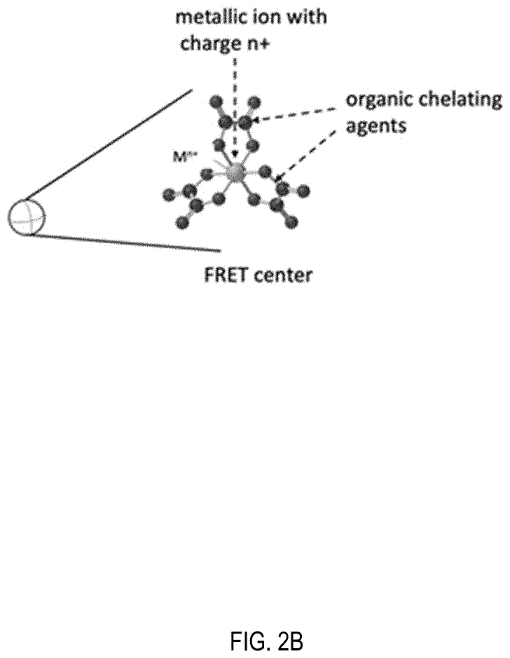

Referring to FIG. 2A, the present invention comprises a ceramic film 10 having multiple pores 16 in combination with fluorescence resonance energy transfer centers 14 disposed throughout the film. Pore wall 12 composition is anodized aluminum oxide (Al₂O₃) ceramic. Pores 16 are ordered in a honeycomb pattern, with depth dimensions not shown at scale. Pores 16 can be through pores or closed pores with an aluminum backing as shown. Each fluorescence resonance energy transfer center 14 comprises a chelated metal ion. Referring to FIG. 2B, fluorescence resonance energy transfer centers 14 may comprise a metal ion chelated by organic molecules.

Acceptable ceramic films with pores are disclosed in Applicant's co-pending U.S. application Ser. No. 16/799,169, hereby incorporated by reference in its entirety. As an example, fabricate a nano-structured aluminum oxide film in which fluorescence enhancement doping centers are not simply contained within the open spaces of the ceramic film nano-pores but are embedded into the aluminum ceramic film to avoid leaching into the environment. Furthermore the chelated FRET center metal is isolated from the quenching effects of water. This result is achieved by means of a chelating anion used to form a doped chelant solution, which is added to the anodizing solution during the process of forming a ceramic film from high purity aluminum foil or plate. The doped ceramic film may contain FRET fluorescence enhancement doping centers (M) and chelating anions (A), such as oxalate, sulfate, cyanate, phosphate, bicarbonate and/or mono and multi-dentate organic ligands, to have the formula

M^{+p}(A⁻ⁿ)ₘ

where M is a metal, a metalloid, a lanthanoid or an actinoid element in any of their oxidation states m that obeys the rule:

n×m≥p

The doped ceramic film may comprise pure chelating anions or a mixture of oxalate, sulfate, cyanate, phosphate, acetates, anions of organic acids such as succinic acid, malonic acid, phosphonates, or phosphinates or a negatively charges mono, di-, tri-, tetra-, penta-, hexa-dentate organic ligand with or without a macro-polycyclic framework, able to impart high thermodynamic and kinetic stability to the suspended anodizing and doped chelant solution mixture. The doping compounds may include a mixture of a metal, a metalloid, a lanthanoid, or an actinoid with group X as follows:

X⁺¹_{q}M^{+p-q}(A⁻ⁿ)ₘ

where X is ammonia, NH₄^{→}, or a Group I alkali element of the series Lithium (Li), Sodium (Na), Potassium (K), Rubidium (Rb) obeying the rule

n×m=p

Optionally NaF and KF salts can be added as the source of alkali ion because fluoride ions bind to the free coordination site of the donor M and help keep away water molecules, which cause quenching of donor luminescence

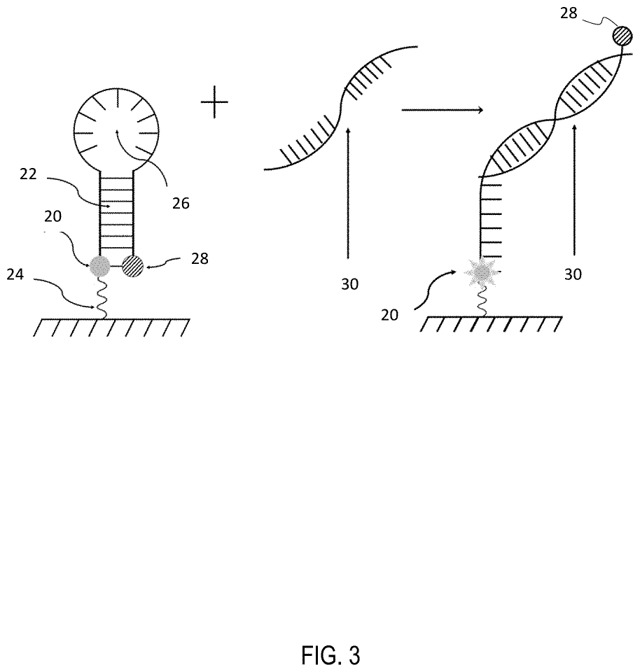

Referring to FIG. 3, the process of DNA hairpin fluorescence upon hybridization may be seen. DNA hairpins are highly specific molecular detection tools, which are the go-to method used to identify specific DNA sequences, gene mutations, or for identifying pathogen RNA in host cells after reverse transcription to DNA. First, a fluorophore molecule 20 is added to a short DNA hairpin sequence 22 engineered from a single strand of DNA and a linker 24 is added on the other side of fluorophore 20 to create a covalent bond to a solid surface. The other end of DNA hairpin sequence 22 contains a quencher 28. The rest of the single strand DNA sequence 26 is chosen to represent the DNA sequence 30 targeted for detection, shown in solution. A typical size for single strand DNA sequence 26 is around 100 base pairs. In the absence of a sequence that is complementary to single strand DNA sequence 26, DNA hairpin sequence 22 keeps quencher 28 and fluorophore 20 in very close proximity, thereby inhibiting fluorescence. In this position and upon external excitation the fluorophore is excited but it does not emit fluorescence, the energy is passed to the quencher molecule and no fluorescence is emitted. However, if a DNA sequence 30 in solution is complementary to the single stand sequence in the hairpin 26, the two strands will hybridize together to form a double strand. The hybridization pulls apart the quencher-fluorophore pair 20 and 28, thereby enabling fluorophore 20 to emit fluorescence upon excitation. At the start of this process, the signal strength is weak due to the early stages of the DNA amplification process. The fluorescence signal can also be weak at the end of the DNA thermal cycling due to extremely low concentrations of the target DNA in the original sample. The present invention provides long lasting (factor of 1000) excitation of the fluorophore which enables amplification of fluorescence and SNR >10

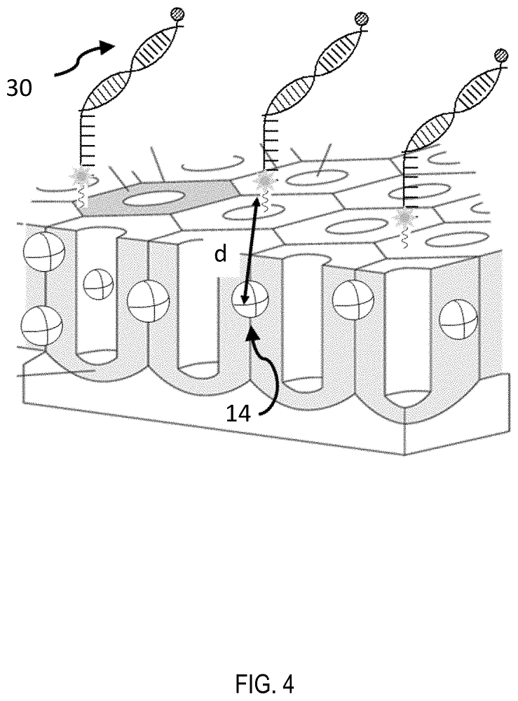

There is seen in FIG. 4, multiple grafted hairpin probes 30 designed as explained above, after hybridization with DNA sequence 30 targeted for detection. The pores of ceramic film 10 are large enough to provide ample space for probes and the PCR reagents (TAQ polymerase, primers, and the DNA sample), to penetrate deep inside the pores. Typical diameter to pore length aspect ratios are 1:1000. Multiple hybridized probes 30 are shown grafted on the surface of the nano-structured ceramic film. However, most of the grafts are within the pores (>100 fold) with sufficient space for DNA amplification during PCR. FRET centers 14 are doped within the ceramic film at low concentrations to avoid self-quenching. The distance (d) between FRET centers and probe fluorophores is maximized to be within 10 nm for optimal signal to noise enhancement.

The photo-active ceramic film can be used in all cases where DNA detection by fluorescence in situ hybridization (FISH) has been utilized. This includes both clinical in vitro diagnostics but also research tools, such as Laboratory Developed Tests (LDTs) which are indispensable in investigations of mutation rates, tracking disease spread and building genomic profiles of diseases. The present invention provides for early detection, such as when a patient sample has a low genetic, pathogenic (bacterial or viral) DNA/RNA charge, such as during the early onset of a disease such as cancer. The present invention also provides for faster and more accurate detection as fewer steps of PCR thermal cycling are needed. The present invention further provides for low volume detection as enhancement occurs within the ceramic discs, which for a typical PCR well top diameter (4.5 mm) this can be as little as ˜10 micro-liters. The present invention additionally provides for low reagent use, which is particularly helpful when TAQ polymerase, primers and probes have limited availability. Finally, the present invention provides for enhancement of SNR with low complexity as the present invention does not require additional instrumentation, reagents, filters or specialized software. The present invention can be configured as a chip to be used with standard fluorescence optical equipment, and thus does not require any additional capital investment. For example, a chip according to the present invention could be used in combination with a conventional real-time PCR system (RT-PCR) to allow for detection of target molecules much faster than with standard substrates due to the improved fluorescent response as the enhanced fluorescence response is provided in real time and with each cycling of the amplification by the RT-PCR system.

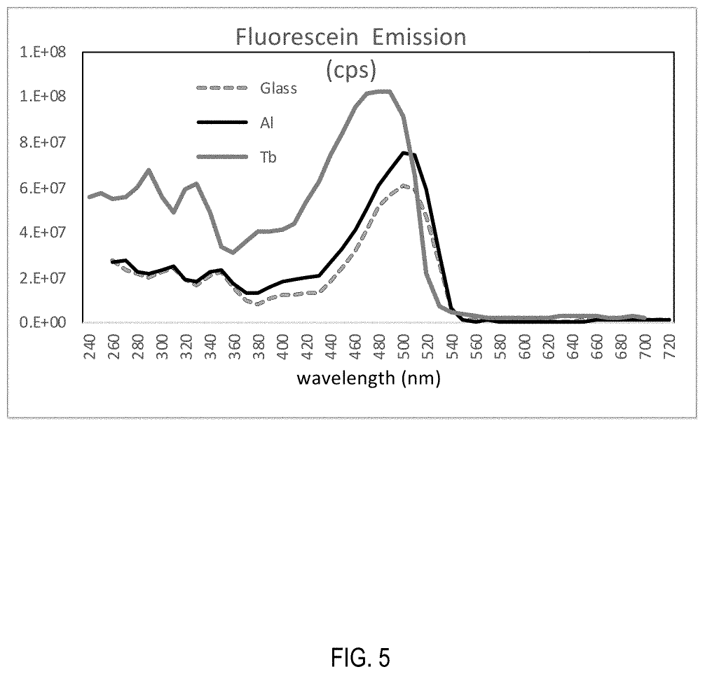

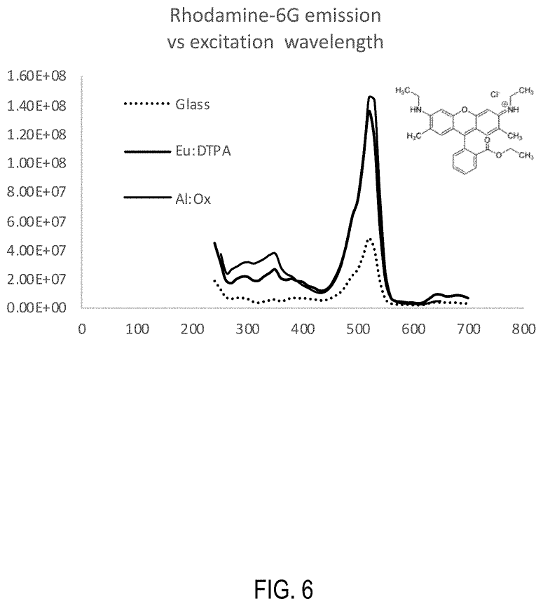

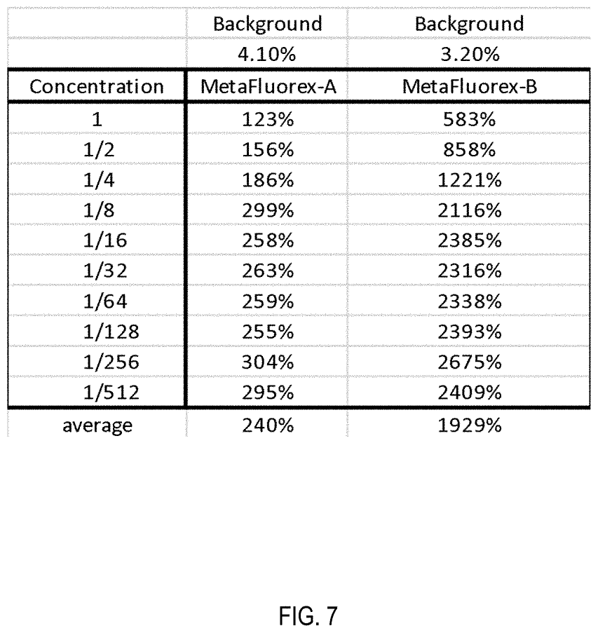

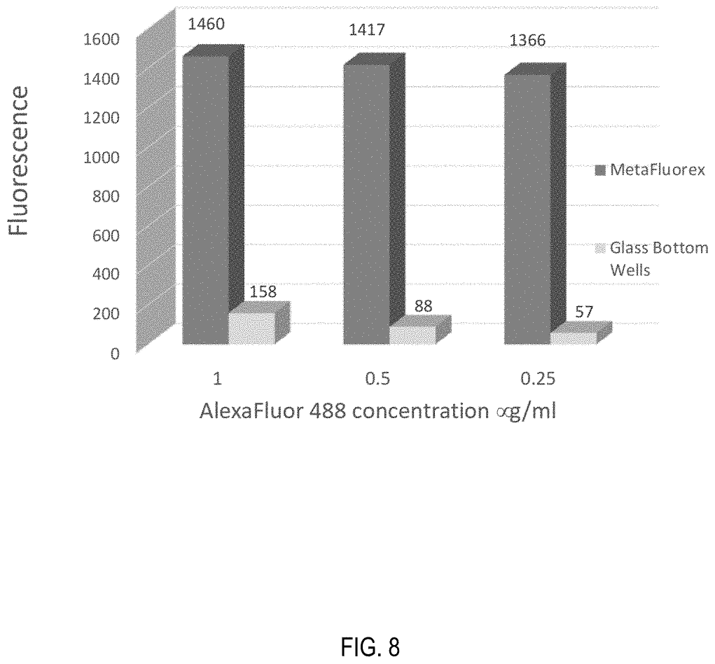

It should be recognized that due to the high increase in fluorescence signals, a one-time recalibration process may be required with positive and negative controls. Also, due to the enhancement beyond the usual FRET enhancement distance, the molecular linker to the fluorescent probe in the hairpin should be approximately 10 nm. FIGS. 5 through 8 show the detection improvement provided by the present invention with respect to various DNA detection fluorophore systems over conventional approaches.

What is claimed is:

1. A substrate for biological detection, comprising
a ceramic film having a plurality of pores formed in a surface of the ceramic film; and
a fluorescence resonance energy transfer center having a chelated metal ion embedded in the ceramic film; and
a hairpin nucleic acid construct covalently linked to the surface of the ceramic film.

2. The substrate of claim 1, wherein the hairpin nucleic acid construct comprises a nucleic acid strand coupled to a fluorophore at one end and a quencher at an opposing end.

3. The substrate of claim 2, wherein the hairpin nucleic acid construct comprises a linker coupled between the fluorophore and the surface of the ceramic film.

4. The substrate of claim 3, wherein the hairpin nucleic acid construct corresponds to a target nucleic acid sequence such that the target nucleic acid construct will hybridize with the nucleic acid strand of the hairpin nucleic acid construct.

5. The substrate of claim 4, wherein the plurality of pores are arranged in a honeycomb pattern.

6. The substrate of claim 5, wherein each of the plurality of pores has a diameter between 50 nm and 400 nm within a tolerance of ten percent.

7. A method of detecting a target nucleic acid strand, comprising the steps of:
providing a ceramic film having a plurality of pores formed in a surface of the ceramic film and a fluorescence resonance energy transfer center having a chelated metal ion embedded in the ceramic film, and a hairpin nucleic acid construct covalently linked to the surface of the ceramic film and comprising a nucleic acid strand coupled to a fluorophore at one end and a quencher at an opposing end; and
exposing the ceramic film to a solution that may contain a target nucleic acid sequence; and
measuring a fluorescence signal from the ceramic film to determine whether the solution containing the target nucleic acid sequence.

8. The method of claim 7, wherein the ceramic film is positioned in a real time polymerase chain reaction (RT-PCR) system.

9. The method of claim 8, wherein the step of measuring the fluorescence signal is repeated.

10. The substrate of claim 9, wherein the hairpin nucleic acid construct comprises a linker coupled to the fluorophore and to the surface of the ceramic film.

11. The substrate of claim 10, wherein the hairpin nucleic acid construct corresponds to a target nucleic acid sequence such that the target nucleic acid construct will hybridize with the nucleic acid strand.

12. The substrate of claim 11, wherein the plurality of pores are arranged in a honeycomb pattern.

13. The substrate of claim 12, wherein each of the plurality of pores has a diameter between 50 nm and 400 nm within a tolerance of ten percent.
